(19) United States
(12) Patent Application Publication
(10) Pub. No.: US 2011/0166674 A1
(43) Pub. Date: Jul. 7, 2011

(54) ANKLE PROSTHESIS

(75) Inventor: Franck Montmartin, Gatey (FR)

(73) Assignee: PROTEOR, Saint-Apollinaire (FR)

(21) Appl. No.: 13/003,223

(22) PCT Filed: Jul. 9, 2009
(86) PCT No.: PCT/FR09/51367

§ 371 Date: Mar. 2, 2011

(30) Foreign Application Priority Data
Jul. 10, 2008 (FR) 08/03930

(51) Int. Cl. A61F 2/66

(52) U.S. Cl. 623/47

(57) ABSTRACT

The invention relates to a prosthetic or orthetic device (1, 1′) of the prosthesis or orthesis articulation module type, particularly for an ankle or ankle foot, comprising: —at least one upper fastening (7′, 7′″) and a lower fastening (7, 7″) connected to one another by at least two link rods (8; 8a, 8b), notably one (8a) at the front and one (8b) at the rear, —at least one articulation connecting the upper fastening (7′, 7′″) and the lower fastening (7, 7″), —at least one drive means (3, 3a, 3b, 5, 5′) connected to a link rod (8; 8a, 8b), secured to the upper fastening (7′, 7″) of the device (1, 1′) and able to move this link rod (8; 8a, 8b) so as to set the lower (7, 7″) and upper (7′, 7′″) fastenings in motion relative to their articulation, in which device the drive means is at least one eccentric (2; 2a, 2b) made to move by means of a cogged wheel (3; 3a, 3b) secured to the eccentric (2; 2a, 2b) and in mesh with an actuator.

The present invention relates to the field of prostheses, notably articulation prostheses and in particular ankle prostheses.

Naturally, ankle prostheses are known in the prior art. For example, U.S. Pat. No. 5,766,264 and PCT patent application WO 2007/0106396 describe an articulated ankle prosthesis comprising passive components with plantar flexion.

The application WO 2006/000211 describes a foot with articulations making it possible to adapt to the situation of the patient, notably with a foot with a statically adjustable or immobile heel. The U.S. Pat. No. 6,966,933 describes a foot prosthesis with an adjustable ankle with different heights of heel.

International PCT applications WO 2005/079712 and WO 2007/027668 describe prostheses of lower limbs making it possible to control the movement of the foot and in particular the movement of the foot/ankle.

The knee prosthesis marketed under the name of “Hydracadence” allows the movement of the foot to be controlled by the movement of the knee.

However, the prostheses of the prior art may turn out to be inadequate notably in terms of extension blocking during the propulsion phase, of raising the tip of the foot in its pendular phase, of adjusting the height of the heel, of quality of the plantar flexion at the contact surface of the heel or else in terms of cost, of weight, of bulk, and even of service life, notably with respect to the quantity of power, for example electric power, necessary for their operation.

These prostheses may therefore have insufficient features relative to the possibilities for adjustment of the ankle relative to the foot and/or to the ground, and in particular relative to the adaptation to the ground when going up and/or when going down, to the dorsiflexion for going up and/or for going down stairs.

The known systems may turn out to be inadequate notably in terms of comfort, of bulk and/or of flexibility. Therefore, the known prostheses may not have a satisfactory comfort, flexibility and/or bulk. It is therefore desirable to provide an enhancement of these features.

There therefore remains a need for ankle or ankle foot prostheses making it possible to completely or partly solve the problems mentioned above.

The object of the invention is therefore to wholly or partly remedy these problems.

Therefore, according to a first aspect, the subject of the present invention is a prosthetic or orthetic device, of the prosthesis or orthesis articulation module type, notably of an ankle or ankle foot, comprising:
at least one upper fastening and a lower fastening, connected to one another by at least two link rods, notably one link rod at the front and one link rod at the rear,
at least one articulation connecting the upper fastening and the lower fastening,
at least one drive means connected to one link rod, secured to the upper fastening of the device and capable of moving this link rod so as to set in motion the lower fastening and upper fastening relative to their articulation, wherein the drive means is at least one eccentric made to move by means of a gearwheel secured to the eccentric and engaged with an actuator.

The eccentric is notably a gearwheel of which the axis of connection to the link rod is offset relative to its axis of rotation.

Said eccentric is made to move notably by a gear or a worm.

In particular, the device is a prosthetic ankle module or a prosthetic ankle foot module intended for tibial or femoral amputees. Most particularly, this module is intended to be incorporated into a prosthesis of a lower limb, notably a foot or tibia.

Therefore, the lower fastening of the device can be connected to a foot prosthesis, orthesis or a portion of a foot prosthesis or orthesis. The upper fastening can be connected with a leg prosthesis or orthesis, notably a prosthesis or orthesis of a lower portion of the leg. This lower fastening can be compatible with various elements of prosthesis or orthesis.

The device according to the invention allows, notably when walking, the lifting movement of the foot during the pendular phase, the adjustment of the height of the heel of the foot, notably via the actuation of the drive means or the rotation of the eccentric, and the adaptation to walking on stairs or on slopes.

“Toward the rear” within the meaning of the present invention means the side closest to the heel of the foot. “Toward the front” within the meaning of the present invention means the side closest to the toe-end of the foot.

The device may comprise at least two link rods and in particular two or four link rods, at least one toward the front and at least one toward the rear. Each link rod can be actuated by an eccentric, itself being linked to a wheel.

The movement of the fastenings relative to one another can be limited in its amplitude by abutments of extension and of flexion. These abutments may also be able to be extended, for example, abutments that are sprung or controlled in movement in order to increase the range of movement of the device.

When the device comprises four link rods, it may comprise one of them on the front left, one on the front right, one on the rear left and the last on the rear right. Most particularly, the two at the front are placed on either side of a front wheel, and the two at the rear are placed on either side of a rear wheel. The front wheel and the rear wheel each form in association with the link rods two eccentrics each actuating one link rod laterally. The assembly may also comprise a mid-plane of symmetry.

The device may comprise at least two eccentrics of which at least one is placed toward the front and at least one is placed toward the rear of the device, interacting with one another. This may have the advantage of reducing the travel, for example of dividing it by two. In other words, in order to make the same movement, this requires less of a rotation or less of an eccentration of the eccentric.

The device may comprise at least two eccentrics front and rear and in particular four eccentrics, and even two front eccentrics and two rear eccentrics.

In the embodiment with at least two link rods, of which one is toward the front and one is toward the rear, when there is contact on the ground, the device can allow plantar flexion without changing the angular position of the eccentrics.

The device also makes it possible to block the articulation in the extension and flexion phase, for example by virtue of at least one rear link rod which butts against the lower fastening.

The instantaneous center of rotation (CIR) can be placed outside the mechanism in a zone close to the axis of physiological articulation of the ankle. This allows plantar flexion when the heel makes contact and allows the range of movement of the device like a physiological ankle.

Other features and advantages of the invention will appear in the detailed description that follows, as an example, of various embodiments of this device. In this description, reference will be made to the appended schematic drawings in which:

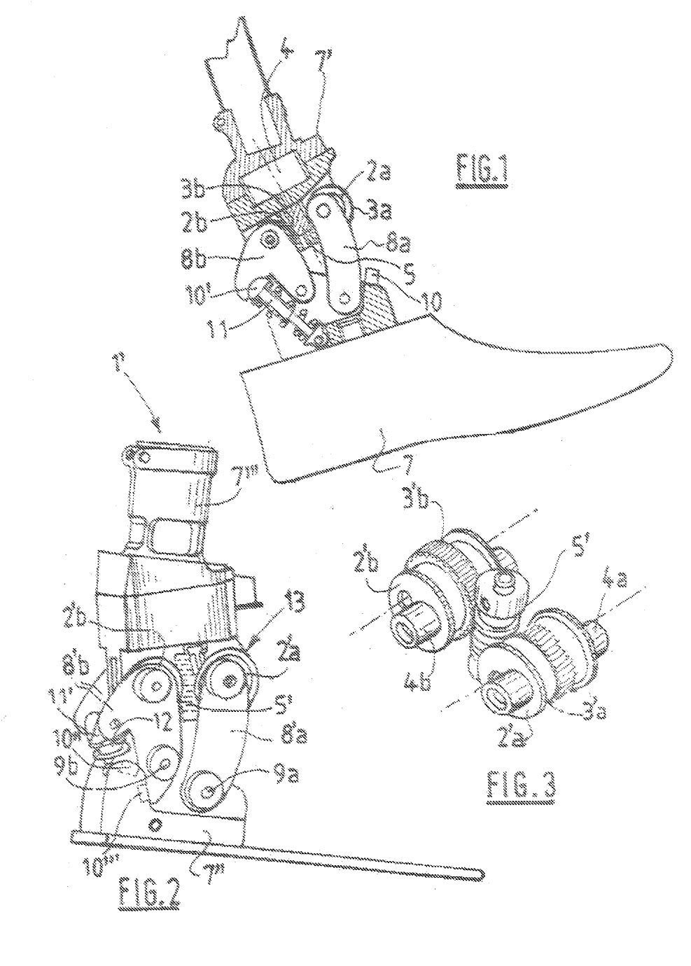

FIG. 1 is a view in section of a device according to the invention, with two link rods of which one is toward the front and one is toward the rear,

FIG. 2 is a view in perspective of a variant embodiment of the device with four link rods, and

FIG. 3 is a view of an eccentric of the device.

FIG. 1 represents a simple device with two link rods of which one 8a is toward the front and one 8b is toward the rear. The device 1 comprises a lower fastening 7 and an upper fastening 7′, which fastenings are connected to one another by the two link rods 8a, 8b, which are in one and the same plane. These link rods are articulated in an articulated parallelogram. The device also comprises two wheel-shaped eccentrics, front and rear 2a, 2b, respectively mounted on the upper fastening and each coupled to a corresponding gearwheel 3a or 3b. These gearwheels are engaged with a middle worm 5, also mounted on the upper fastening 7′ and making it possible to actuate each of the opposite gearwheels by means of a motor 4. The eccentrics 2a, 2b (opposite one another) are each present on the axis of an associated front gearwheel 3a, rear gearwheel 3b. They are each mounted coaxially on a gearwheel. The link rods are each connected to the adjoining gearwheel via an axis of articulation offset from the axis of rotation of the gearwheel, and parallel to the latter, which allows the fastenings 7, 7′ a range of movement relative to one another.

The device comprises a front abutment 10 making it possible to limit the movement of the front link rod toward the front and blocking it.

The device also comprises a rear sprung system 11 with telescopic column forming a rear abutment 10′. It makes it possible to damp the force when the heel contacts the ground. This sprung system 11 also makes it possible to return the articulation to the extension position. Without this system, the foot would be free about its axis of rotation.

FIG. 2 represents a device 1′ with four eccentrics 2′a, 2′b in wheel form, notably front/rear and left/right, of which two can be seen in the figure and with two associated gearwheels 3′a, 3′b that can be seen in FIG. 3. It may comprise one or two front eccentrics 2′a and one or two rear eccentrics 2′b, or even more. It may comprise two or four link rods 8′a, 8′b connecting the lower fastening 7″ and upper fastening 7′″. Two opposite link rods front and rear may be in one and the same plane and be articulated in an articulated parallelogram.

The device also comprises a rear flexion abutment 10′″ and a rear extension abutment 10″ making it possible to limit the movement of the rear link rod 8′b and blocking it.

The device may also comprise a sprung system 11′ with telescopic column also making it possible to damp the force when the heel contacts the ground. This sprung system 11′ also makes it possible to return the articulation to the extension position. This system may comprise an adjustment thumbwheel 12 in order to allow the users to adapt the flexibility of the articulation when the heel makes contact.

The lower fastening 7 or 7″ of the device can be connected to a foot prosthesis or orthesis (not shown). The upper fastening 7′ or 7′″ can be connected to a leg prosthesis or orthesis, notably of a lower portion of the leg (not shown). The lower fastening can be compatible with various standard models of prosthetic feet, for example sachs feet or energy-restoration feet or prosthesis or orthesis elements.

FIG. 3 represents more particularly the portion 13 of a device actuating the eccentrics 2′a, 2′b of FIG. 2. It applies similarly to the device of FIG. 1. It comprises the gearwheels 3′a, 3′b and the worm 5′, the latter making it possible to actuate the gearwheels 3′a, 3′b and hence the eccentrics 2′a, 2′b.

The device may comprise one or two gearwheels 3′a, 3′b connected to the eccentrics 2′a, 2′b (FIG. 3), a front gearwheel 3′a and a rear gearwheel 3′b. The front gearwheel 3′a is mounted coaxially with each of the front eccentrics placed on either side of the latter and comprises a front spindle 4a. The rear gearwheel 3′b is mounted coaxially with each of the rear eccentrics placed on either side of the latter and comprises a rear spindle 4b. The front spindle 4a and rear spindle 4b are connected to the front and rear link rods respectively. These spindles are offset from the axis of rotation indicated by a dot and dash line.

The front gearwheel 3′a and rear gearwheel 3′b are connected with symmetry relative to a longitudinal midplane of the device to the front link rod 8′a and rear link rod 8′b which can be connected in twos to the lower fastening 7″ respectively via a front articulation spindle 9a and a rear articulation spindle 9b.

The actuator may notably be a worm 5′, a gear, a belt pulley or a bevel gear (not shown). The actuator is coupled to the gearwheels 3′a, 3′b. A motor (not shown) can be used to rotate this worm 5′.

In particular, the worm 5′ is always in contact with the gearwheels 3′a, 3′b; it sustains a force in the plantar flexion phase. In this case, the gearwheel 3′a, 3′b/worm 5′ system is irreversible: the worm rotates the wheel but not vice versa.

The front eccentric 2′a may have an eccentration ranging from 1 to 10 mm, from 3 to 8 mm, and even approximately 5 mm. This may notably allow a movement of 2 to 20 mm, in particular of 6 to 16 mm, and even approximately 10 mm on a half-turn. The total angular travel of the front eccentric 2′a can range up to 180°.

The rear eccentric 2′b may have a rear eccentration ranging from 1 to 10 mm, in particular from 3 to 8 mm, and even approximately 5 mm. This may notably allow a movement of 2 to 20 mm, in particular of 6 to 16 mm, and even approximately 10 mm on a half-turn. The total angular travel of the rear eccentric 2′b can range up to 180° in a similar manner.

The device can allow, when walking, an angle of flexion ranging from 2 to 30°, notably from 5 to 20°, in the dorsiflexion phase (pendular phase).

It may also allow an angle of flexion ranging from 1 to 40°, notably 5 to 30° in the plantar flexion phase (foot contact).

This prosthesis may also allow a height adjustment of the heel ranging from 0 to 100 mm, notably from 10 to 60 mm.

The device may comprise position, pressure and/or acceleration sensors (not shown) making it possible to control the actuator in order to raise the tip of the foot, notably during the pendular phase, or else other plantar flexion or dorsiflexion movements, both during the pendular phase and during the phase of contact with the ground. These sensors can be connected to a system for commanding and controlling the actuator for the purpose of controlling the movement of the device when walking.

Finally, as mentioned above, the instantaneous center of rotation (CIR) is placed outside the mechanism, in a zone close to the physiological axis of articulation of the ankle and slightly in front of the latter. The instantaneous center of rotation (CIR) moves during the movement through a short segment, preferably curved (not shown). This allows plantar flexion during the contact of the heel, reducing the power necessary for the flexion forces, and allows the range of movement of the device like a physiological ankle.

1. A prosthetic or orthetic device, of the prosthesis or orthesis articulation module type, notably of an ankle or ankle foot, comprising:
at least one upper fastening and a lower fastening, connected to one another by at least two link rods, notably one link rod at the front and one link rod at the rear,
at least one articulation connecting the upper fastening and the lower fastening,
at least one drive means connected to one link rod, secured to the upper fastening of the device and capable of moving this link rod so as to set in motion the lower fastening and upper fastening relative to their articulation, wherein the drive means is at least one eccentric made to move by means of a gearwheel secured to the eccentric and engaged with an actuator.

2. The device as claimed in claim 1, characterized in that the eccentric is a gearwheel of which the axis of connection to the link rod is offset relative to its axis of rotation.

3. The device as claimed in claim 1, characterized in that the lower fastening of the device can be connected to a foot prosthesis, orthesis or a portion of a foot prosthesis or orthesis and the upper fastening can be connected with a leg prosthesis or orthesis, notably a prosthesis or orthesis of a lower portion of the leg.

4. The device as claimed in claim 1, characterized in that it comprises at least two eccentrics front and rear, and in particular four eccentrics, and even two front eccentrics and two rear eccentrics.

5. The device as claimed in claim 4, characterized in that the front eccentric has an eccentration ranging from 1 to 10 mm, in particular from 3 to 8 mm, and even approximately 5 mm.

6. The device as claimed in claim 4, characterized in that the rear eccentric has an eccentration ranging from 1 to 10 mm, in particular from 3 to 8 mm, and even approximately 5 mm.

7. The device as claimed in claim 4, characterized in that the total angular travel of the front eccentric can range up to 180°.

8. The device as claimed in claim 4, characterized in that the total angular travel of the rear eccentric can range up to 180°.

9. The device as claimed in claim 1, characterized in that said actuator is at least one worm coupled to the gearwheel, said worm being coupled to a motor making it possible to turn it.

10. The device as claimed in claim 1, characterized in that it comprises position, pressure and/or acceleration sensors making it possible to control the drive means or the actuator in order to control the movement of the device.

11. The device as claimed in claim 1, characterized in that the movement of the fastenings relative to one another is limited in its amplitude by extension and flexion abutments.

12. The device as claimed in claim 1, characterized in that it comprises extendable abutments, for example abutments that are sprung or controlled in movement in order to increase the range of movement of the device.

13. The device as claimed in claim 1, characterized in that it comprises an instantaneous center of rotation placed outside the mechanism, in a zone close to the physiological axis of articulation of the ankle.
